(19) United States
(12) Patent Application Publication
(10) Pub. No.: US 2014/0228784 A1
(43) Pub. Date: Aug. 14, 2014

(54) Negative Pressure Device for Treating Wounds

(71) Applicants: Paolo Mazzoni, Modigliana (IT); Tommaso Bianchi, Bologna (IT)

(72) Inventors: Paolo Mazzoni, Modigliana (IT); Tommaso Bianchi, Bologna (IT)

(73) Assignee: MET S.R.L., San Lazzaro Di Savena (IT)

(21) Appl. No.: 14/119,751

(22) PCT Filed: May 24, 2012
(86) PCT No.: PCT/IB2012/052619

§ 371 Date: Feb. 4, 2014

(30) Foreign Application Priority Data
May 24, 2011 (EP) BO2011A000296

(51) Int. Cl. A61M 35/00; A61F 13/02; A61M 1/00

(52) U.S. Cl. CPC A61M 35/00; A61M 1/0027; A61M 1/0025; A61M 1/0035; A61F 13/0216; A61F 2013/0028
USPC 604/305

(57) ABSTRACT

A device for treating wounds with negative pressure is provided that includes an adhesive film that defines a chamber in relation to the wound; a vacuum source can be adjusted to produce negative pressures in the same chamber; a canister for collecting liquids drained from the wound; a reservoir containing medicated liquids to be instilled in the wound; an unit for setting both the negative pressure and the quantity of administered medication; and a pressure sensor to detect the negative pressure present in said chamber. The device further includes a spiral diffuser mounted on said film comprising a high number of protrusions on tube coils, the spiral diffuser being connected both to vacuum source and to a pump; micro-valves for diffusing medicated liquids, one for each protrusion on coiled tube of spiral diffuser; and a pump administering medicated liquids exerting a pressure capable of opening the micro-valves present in protrusions.

### TECHNICAL FIELD

The present invention tackles the problem of treating wounds, in particular wide wounds or chronic wounds and pressure sores.

### BACKGROUND ART

The problem of treating this type of wounds is strongly felt, because population aging and the present epidemics of obesity and diabetes have increased the number of people affected. Moreover, the number of antibiotic-resistant microorganisms is increasing, and this kind of therapy is loosing efficacy. National health services have less and less resources, and therefore all the interventions facilitating and accelerating the procedures performed by health operators for treating wounds are welcome, especially systems allowing to perform wound treatment at patient's home instead of hospital treatment.

From WO 9309727 devices applying a pressure lower than chamber pressure to wounds are known. In this field, “reduced pressure”, “vacuum treatment”, “treatment under negative pressure” are synonyms.

In particular, WO 9309727 describes a device applying negative pressure to a wound. The device is inserted on the wound surface, and the wound sealed with a polymeric sheet adhering to the skin. Between the sheet and the wound surface a open-cell polymeric foam is inserted (screen means) in order to avoid wound hypertrophy. A problem linked to this solution is that tissues tend to grow within the polymeric foam itself, therefore when the dressing is changed, the patient feels a strong pain. The removal of the polymeric foam during dressing change can cause strong bleeding. Moreover, removing all the polymeric foam from the wound is not easy, and polymeric foam left in the wound can cause dangerous infections. These problems constitute the ground for a notification issued by FDA on Nov. 13, 2009 (http://www.fda.gov/MedicalDevices/Safety/AlertandNotices/ PublicHealthNotifications/ucm190658.htm), wherein 6 deaths and 77 injuries associated with Negative Pressure Wound Therapy systems are reported. Retention of foam dressing pieces and foam adhering to tissues or imbedded in the wound were noted in 32 injury reports.

Finally, said screen means are not easy to place in the wound: a certain manual ability is requested by the health operator cutting the polymeric material in a shape fit for the wound, and a considerable part of the time needed for dressing is spent in fitting such material to the wound shape.

In WO 2004/037334 and U.S. Pat. No. 4,382,441 devices are described which, in addition to applying a negative pressure to the wound so as to drain liquids (exudates), can also irrigate the wound with liquids accelerating healing. In particular, in WO 2004/037334 inflatable hollow bodies are described, to be inserted between the wound and the wound sealing means.

In WO 2009/002260 a device for treating wounds with reduced pressure is described, comprising, as the already cited devices, a wound sealing film, a reduced pressure source, as well as a tube, characterized in that the interior of the tube comprises a longitudinal first strand made of a hydrophobic material, as well as a second strand, made of an open-pored hydrophilic material, extending longitudinally over at least a part of the length of the tube. Said material strands are enclosed in a tube casing made of a flexible material. The fluid-receiving element can be formed by rolling-up or folding of a long portion of the tube (FIG. 14).

### DISCLOSURE OF INVENTION

Aim of the present invention is to provide a wound treatment device that can at the same time both establish a reduced pressure in the wound, and irrigate the wound with liquids and/or gases capable to accelerate its healing.

A second aim of the present invention consists in providing a device not needing screen means to be inserted between the wound and the tube applying vacuum in order to avoid wound hypertrophy.

A further aim of the present invention consists in providing a simplified device allowing to perform wound treating at home, facilitating as much as possible wound dressing to the health operator, decreasing the time necessary for dressing.

The whole device applied to the patient comprises:
a vacuum source producing a negative pressure adjustable between 0 and at least −200 mm Hg, so that pressure increases very gradually, without causing pain to the patient;
a pump administering medication of different nature (e.g. antibiotics, pain-relievers, anti-inflammatory, cellular proliferation stimulants, dermal matrix components, vascular growth factors, etc.); the pump must be able to exert a pressure at least superior to 1 bar;
a spiral diffuser, at the same time on one side diffusing medications in an uniform way with dosages inferior even to 1 ml, and on the other side suctioning so that the wound is kept under negative pressure; said spiral diffuser is connected on one side to the vacuum source, and on the other to the pump administering medicaments;
an adhesive polyurethane film to seal and isolate the wound.

The vacuum source is a membrane pump electronically controlled through a sensor detecting the value of negative pressure. The pump is connected to a canister collecting the fluids drained from the wound; in the tube an antibacterial filter is inserted. In the canister there is a gelling agent converting the fluids drained from the wound into a gel, having a more agreeable aspect for the patient and necessary for the norms concerning hospital waste management. In the canister a tube coming from the wound, and another tube going to the vacuum source are inserted. A micro-hole in the sealing system allows a continuous flow of liquids and gas in the suction tube, avoiding the formation of stasis in the tube loops and controlling the functioning of the vacuum system. Preferably, the value of the negative pressure is not steady, but varies between two values set by the operator (e.g., −110 mm Hg to −130 mm Hg).

The pump for administering medication can be, for instance, a peristaltic volumetric pump administering a liquid volume varying between 0,1 ml/min and 5 ml/min. It is electronically controlled and can administer the volumes set by the operator in a given time (e.g. 1 ml every 2 hours).

The spiral diffuser is made of a rolled up flexible tube made of polymeric material, that can be unrolled and cut in order to fit it to wound dimensions. The tube exhibits hollow villi; in each villus a valve is present, through which the medication is administered. It is important to note that the valve present in each villus is normally closed and opens only when a positive pressure is exerted by the pump for administering medications. The micro-valves are closed if the negative pressure caused by the vacuum source or chamber pressure is acting. In this way very small quantities of medication can be administered, with the certainty to wet wound surface. At the center of the spiral diffuser there is the end of the suction tube, and the micro-hole allowing a constant active pressure flow. A pH-sensor is moreover present.

The polymeric polyurethane film is adhesive and able to adhere to the healthy wound borders. This guarantees vacuum in the wound.

### BRIEF DESCRIPTION OF THE DRAWINGS

The present invention will now be described in detail with the help of the following figures, showing:

FIG. 1A and B a schematic view of the spiral diffuser inserted in the wound;

FIG. 2 the components of the device;

FIG. 3 a block diagram of the device;

FIG. 4 A and B detailed views of the spiral diffuser;

FIG. 5 an inferior view of diffuser 11;

FIG. 6 a vertical section of diffuser 11.

### BEST MODE FOR CARRYING OUT THE INVENTION

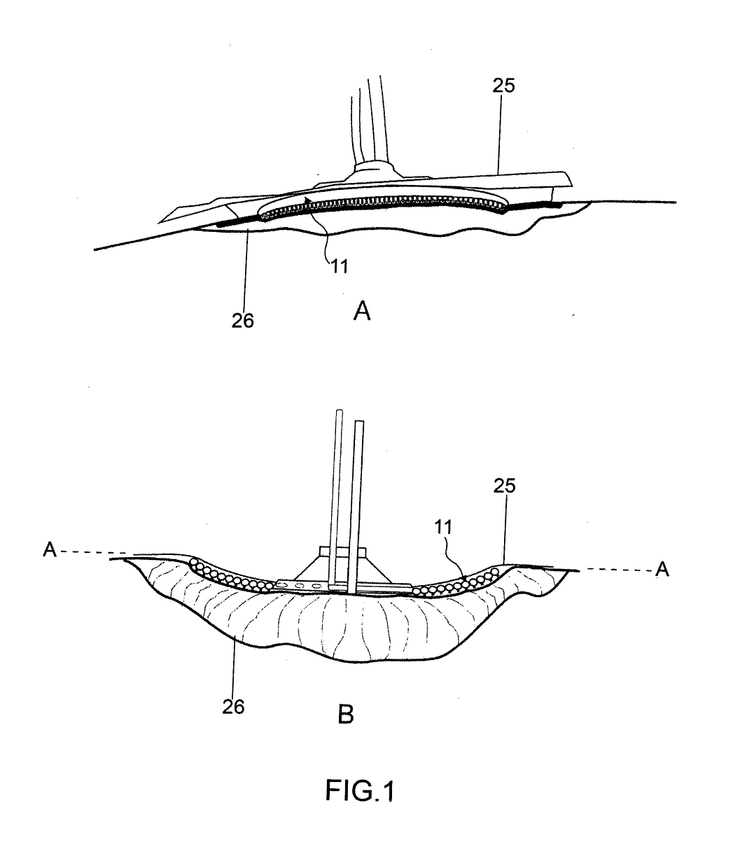

FIG. 1 shows the positioning of spiral diffuser 11 in wound 26; spiral diffuser 11 is covered by polymeric polyurethane film 25 sealing wound 26.

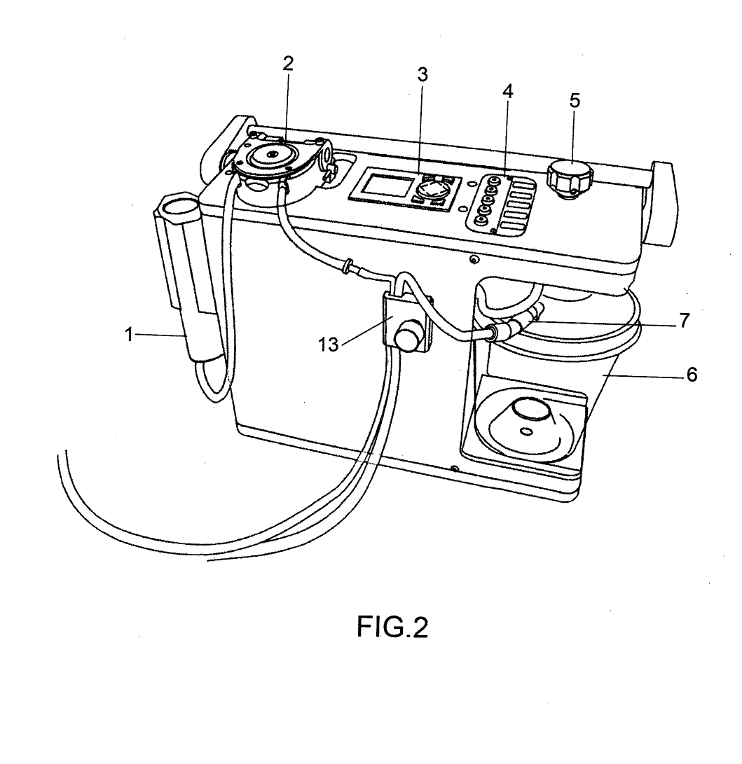

FIG. 2 shows all the parts of the device: irrigating liquid reservoir 1, peristaltic pump 2, unit 3, with display, for programming the device, touches 4, knob 5 for blocking the drained liquid canister, canister 6 for liquids drained from wound 26, connection 7 to the vacuum pump, clamp 13 for single-use tubes.

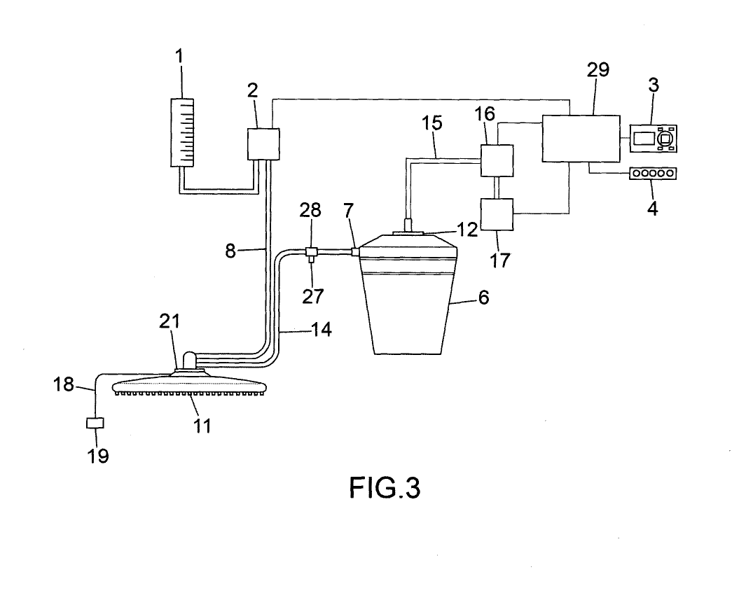

FIG. 3 is a block diagram of the device, wherein 1 is the irrigating liquid reservoir, from which peristaltic pump 2 draws medicated liquid, sending it to spiral diffuser 11 through tube 8. The instilled medication can also be a liquid containing a gas.

The volume of instilled liquid and instillation times are programmed through unit 3. The wound (not shown in FIG. 3) is kept under negative pressure through tube 14, which is connected to liquid-collecting canister 6 through connection 7. Indicatively, the drained liquid collecting canister can contain 800 ml. Negative pressure is generated through pump 16, connected to liquid-collecting canister 6 through tube 15 and antibacterial filter 12. The value of negative pressure is controlled through pressure sensor 17 and an electronic board 29. Once a given value of negative pressure is set, pressure sensor 17 will control the pump, through the electronic board 29, so that on canister 6, tube 7 and spiral diffuser 11 the desired level of negative pressure is obtained.

The quantity of medicated liquid administered must be very precise, and this is obtained through peristaltic pump 2 which is a volumetric pump, that at every turn of its axle can transfer a very small volume of liquid, in a very precise way. Pump 2 is controlled through programming panel 3, and at every turn can pump a minimum of 0,1 ml. The system can control the pump every half turn, so that the minimum administrable quantity is 0,05 ml. By controlling the number of turns and the speed of the pump, it is also possible to instill big quantities of liquid, for instance for cleansing the wound (e.g. 500 ml every 20 minutes). Suction manifold 21 is moreover connected to a capillary tube 18 at the end of which an antibacterial micro-filter 19 is present.

In this way a minimal quantity of atmospheric air can enter, so that a very small air flow is formed in tube 14, in order to avoid stasis formation in tube loops and to control the vacuum system, varying the value of negative pressure in a time controlled by the electronic system, which can detect an occlusion or a leakage in the system. On tube 14 an optical sensor 28 can also be inserted, detecting blood presence in the drained liquids. If blood in drained liquids is over a threshold level, this means that a bleeding is occurring. Vacuum pump then stops and the wound is automatically brought back to chamber pressure; moreover an acoustic/optic alarm is activated informing patient and health operator of the bleeding. Optionally also a sensor 27 detecting the status of the wound can be present, showing it on the unit display 3. The wound status can be communicated to the physician by a home-treated patient, too. Sensor 27 can be e.g. a pH-meter, analyzing liquids drained from the wound. In this case, too, alarms can be activated.

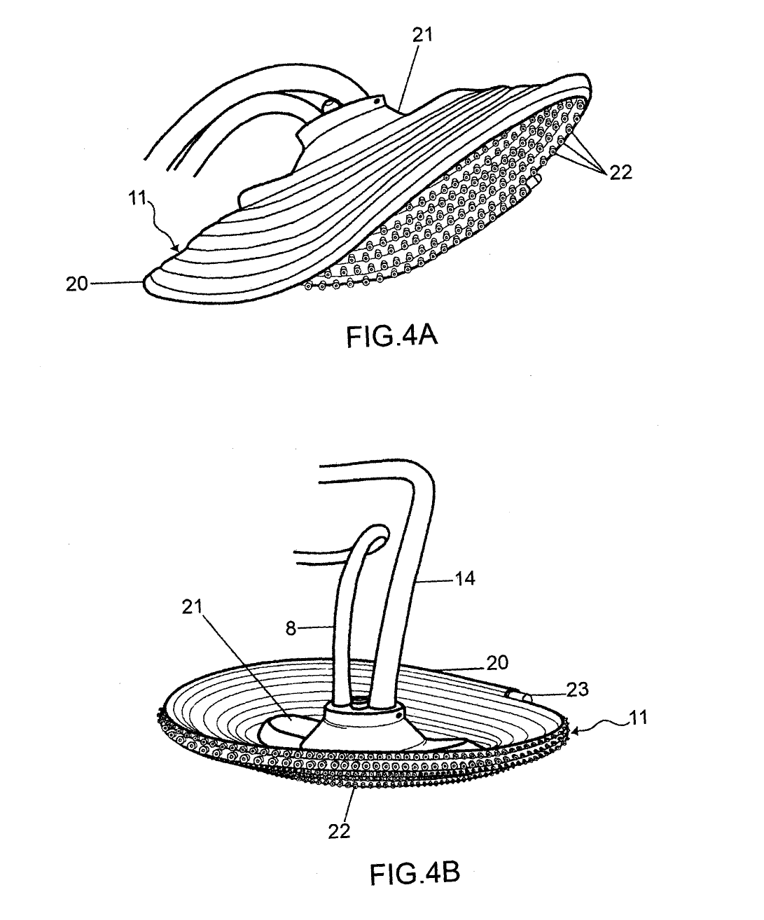

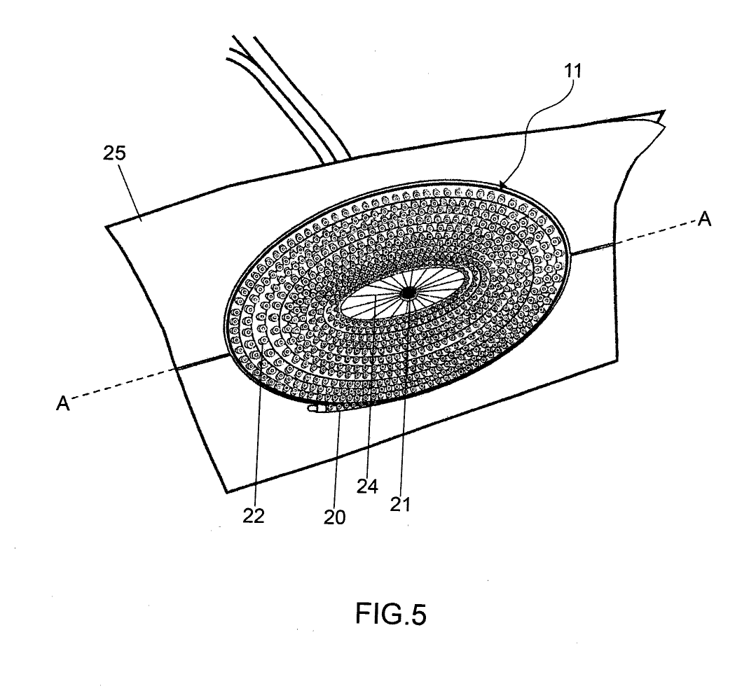

FIGS. 4 and 5 show in greater detail spiral diffuser 11. Spiral diffuser 11 consists in a coiled tube 20 which can be unrolled and cut according to wound dimensions. The coils of tube 20 are tangentially connected to each other; when, in order to reduce the dimensions of spiral diffuser 11, tube 20 is unrolled, the external coil must be detached from the internal coil. Once the desired dimension of spiral diffuser 11 is determined by cutting the exceeding tube, tube 20 is closed at its distal end with a plug 23. 21 indicates the suction manifold in its entirety, which is internally connected to tube 14. The suction manifold 21 exhibits grooves 24 in order to distribute negative pressure on the wound bed. Tube 20 shows on its inferior surface a very high number of protrusions 22 (e.g., a protrusion every 2 mm). Such protrusions 22 have a tapered shape with an external maximal diameter of about 1,5 mm and a depth of about 1,5 mm. Roughly, at least 9 protrusions should be present for each square centimeter.

The hole in the protrusion under chamber pressure and negative pressure is closed, and opens only under the positive pressure exerted by pump 2 in order to administer medicated liquids. The protrusions moreover increase the contact surface between the spiral diffuser 11 and wound 26.

FIG. 5 shows an inferior view of spiral diffuser 11, which can be subdivided into two distinct areas: a first central area 21 and a second area, forming an annulus around the first area, made by tube 20 coils. Grooves 24 in central area 21 distribute negative pressure in a more diffused way on the wound bed. Tube 20, instead, actively instills medications through protrusions 22. Protrusions 22, moreover, further diffuse negative pressure on wound bed and increase the surface in contact with the wound, favoring its healing.

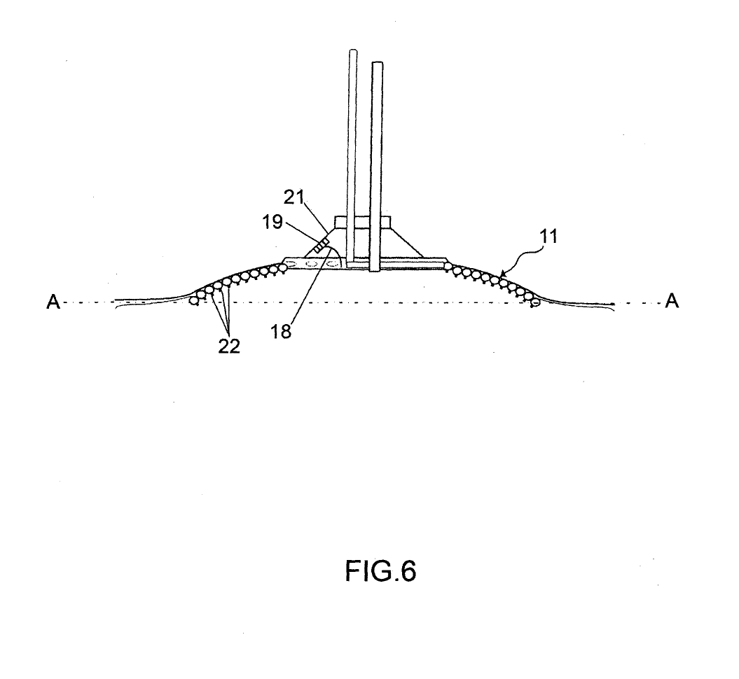

In FIG. 6 a vertical section of spiral diffuser is shown according to line A-A shown in FIG. 5. The proximity of coils in the spiral diffuser, the partial connection between coils, the free space between coils, all contribute to the diffusion of vacuum on all the wound bed, as shown in FIG. 5.

In the section of suction manifold 21 capillary tube 18 is visible, communicating with environment through antibacterial filter 19.

In an alternative embodiment, tube 8 can be connected to a device administering gas under pressure, e.g. oxygen (O₂) or oxygen/ozone mixtures (O₂/O₃). In this case, tube 8 and tube 20 are filled with gas.

1. Device for treating wounds with negative pressure, comprising:
an adhesive film (25) that defines a chamber in relation to the wound;
a vacuum source (16) can be adjusted to produce negative pressures in the same chamber;
a canister (6) for collecting liquids drained from the wound;
a reservoir (1) containing medicated liquids to be instilled in the wound;
an unit (3) for setting both the negative pressure and the quantity of administered medication;
a pressure sensor (17) to detect the negative pressure present in said chamber; characterized in that the device further it comprises
a spiral diffuser (11) mounted on said film comprising a high number of protrusions (22) on tube (20) coils, the spiral diffuser (11) being connected both to vacuum source (16) and to a pump (2);
micro-valves for diffusing medicated liquids, one for each protrusion (22) on coiled tube (20) of spiral diffuser (11); and
a pump (2) administering medicated liquids exerting a pressure capable of opening the micro-valves present in protrusions (22).

2. Device according to claim 1, wherein the micro-valves in protrusions (22) are closed under negative pressure and room pressure, and open only when in tube (20), through pump (2), a sufficient pressure is exerted.

3. Device according to claim 2, moreover comprising in suction manifold (21) a capillary tube (18), at the end of which an antibacterial micro-filter (19) is present.

4. Device according to claim 2, wherein tube (8) must be connected to a device administering gas or gas mixtures under pressure, with controlled flow.

5. Device according to claim 2, further comprising a sensor (27) detecting the status of the wound.

6. Device according to claim 2, further comprising an optical sensor (28) detecting the presence of blood in drained liquids exceeding a given threshold.

7. Device according to claim 2, further comprising a vacuum sensor (17) detecting the pressure in the wound.

8. Device according to claim 1, further comprising at least one of a sensor (27) detecting the status of the wound, an optical sensor (28) for detecting the presence of blood in drained liquids exceeding a given threshold, and a vacuum sensor (17) for detecting the pressure in the wound, and wherein each sensor can activate an alarm and stop the device when safety threshold is exceeded.

9. Device according to claim 5, wherein the sensor (27) is adapted to detect the pH of the wound.
